(19) United States
(12) Patent Application Publication
(10) Pub. No.: US 2007/0293706 A1
(43) Pub. Date: Dec. 20, 2007

(54) Synthesis Of 1,5-Disubstituted-2-Hydroxy-Gibbatetraen-6-Ones

(76) Inventors: Robert R. Wilkening, Maplewood, NJ (US); Amy Fried, San Diego, CA (US); Dann L. Parker JR., Cranford, NJ (US)

Correspondence Address: MERCK AND CO., INC, P O BOX 2000, RAHWAY, NJ, 07065-0907, US

(21) Appl. No.: 11/666,318

(22) PCT Filed: Oct. 28, 2005
(86) PCT No.: PCT/US05/39573

§ 371 Date: Apr. 25, 2007

Related U.S. Application Data
(60) Provisional application No. 60/623,911, filed on Nov. 1, 2004.

(51) Int. Cl. C07C 45/61; C07C 49/755

(52) U.S. Cl. 568/322

(57) ABSTRACT

1,5-disubstituted-2-hydroxy-gibbatetraen-6-ones are useful as estrogen receptor modulators and as precursors to estrogen receptor modulators. The current invention provides a method for the synthesis of 1,5-disubstituted-2-hydroxy-gibbatetraen-6-ones from simple indanone starting materials via a Robinson-type annulation followed by an internal alkylation reaction. This invention further describes the novel use of a fluoroethyl substituent as a latent alkylating group for an internal cyclization reaction.

### BACKGROUND OF THE INVENTION

Naturally occurring and synthetic estrogens have broad therapeutic utility, including: relief of menopausal symptoms, treatment of acne, treatment of dysmenorrhea and dysfunctional uterine bleeding, treatment of osteoporosis, treatment of hirsutism, treatment of prostatic cancer, treatment of hot flashes and prevention of cardiovascular disease. Because estrogen is very therapeutically valuable, there has been great interest in discovering compounds that mimic estrogen-like behavior in estrogen responsive tissues.

1,5-disubstituted-2-hydroxy-gibbatetraen-6-ones are useful as estrogen receptor modulators and as precursors to estrogen receptor modulators. The current invention provides a method for the synthesis of 1,5-disubstituted-2-hydroxy-gibbatetraen-6-ones from simple indanone starting materials via a Robinson-type annulation followed by an internal alkylation reaction. This invention further describes the novel use of a fluoroethyl substituent as a latent alkylating group for an internal cyclization reaction.

### SUMMARY OF THE INVENTION

By this invention, there are provided processes for the preparation of compounds of structural formula I:

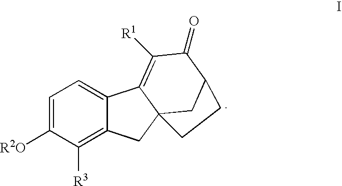

### DETAILED DESCRIPTION OF THE INVENTION

By this invention, there are provided processes for the preparation of compounds of structural formula I:

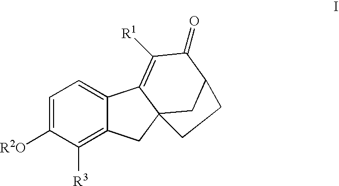

comprising the steps of:
a) Reacting a 2-substituted indanone of formula II with methyl vinyl ketone in the presence of a base to form a diketone of formula III; 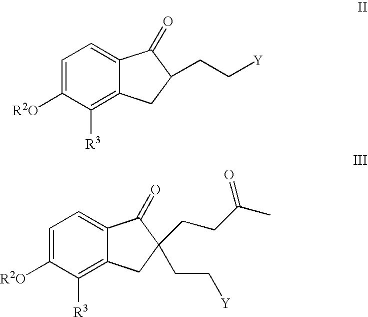
b) Cyclizing the diketone of formula III to form a tetrahydrofluorenone of formula IV; 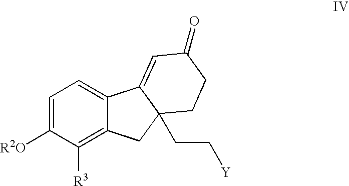
c) Performing an internal alkylation reaction to form a bridged tetrahydrofluorenone of formula V; 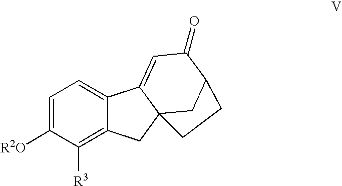
d) Substituting the enone double bond of the bridged tetrahydrofluorenone of formula V to yield the compound of formula I;
   wherein R¹ is fluoro, chloro, bromo, iodo, cyano, C₁₋₄ alkyl, C₂₋₄ alkenyl, C₂₋₄ alkynyl, C₃₋₆ cycloalkyl, aryl, or heteroaryl, wherein said alkyl, alkenyl, alkynyl, cycloalkyl, aryl and heteroaryl groups are optionally substituted with one, two or three groups selected from the group consisting of fluoro, chloro, bromo, iodo, cyano and OR^{a};
   R² is hydrogen, R^{a}, (C═O)R^{a}, (C═O)OR^{a};
   R³ is hydrogen, fluoro, chloro, bromo, iodo, C₁₋₂ alkyl, cyano or OR^{a};
   Y is fluoro, chloro, bromo, iodo, methanesulfonyloxy, p-toluenesulfonyloxy, trifluoromethanesulfonyloxy, or a precursor thereof;
   R^{a} is hydrogen, C₁₋₄ alkyl or phenyl.

Y is defined as fluoro, chloro, bromo, iodo, methanesulfonyloxy, p-toluenesulfonyloxy, trifluoromethanesulfonyloxy, or a precursor thereof. Precursors include hydroxyl or protected hydroxyl. Suitable protecting groups for hydroxyl are known to those skilled in the art.

In an embodiment of the invention, R² is hydrogen, R^{a}, (C═O)R^{a}, (C═O)OR^{a} or a protecting group for a phenolic hydroxyl.

A 2-substituted indanone of formula II is reacted with methyl vinyl ketone in the presence of a base to form a diketone of formula III. In an embodiment of the invention, the base includes, but is not limited to sodium methoxide in methanol, potassium hydroxide in ethanol and DBU in THF.

The diketone of formula III is cyclized to form a tetrahydrofluorenone of formula IV. This cyclizing step is performed under acidic or basic conditions. In an embodiment of the invention, appropriate basic conditions include, but are not limited to: sodium hydroxide in ethanol, sodium methoxide in methanol, and pyrrolidine-acetic acid in toluene. In an embodiment of the invention, appropriate acidic conditions include, but are not limited to: hydrochloric acid in acetic acid, trifluoroacetic acid, and p-toluenesulfonic acid in toluene.

A bridged tetrahydrofluorenone of formula V is formed by an internal alkylation reaction. This reaction is performed in the presence of an organic base, performed with heating or preformed in the presence of an organic base with heating. For the case when Y is fluoro, suitable conditions for this cyclization include, but are not limited, to LiCl in DMF at 150° C. or KN(TMS)₂ in THF from −78° C. to 25° C. In a class of the invention, when Y is fluoro, the reaction is performed in the presence of an organic base with heating, wherein the organic base is LiCl in DMF and heated at 150° C. Alternatively, the fluoroethyl substituent of IV (Y═F) can be first converted to a more reactive bromoethyl substituent (Y═Br) by treatment of IV with BBr₃ in CH₂Cl₂ from −78° C. to 25° C. Compound IV (Y═Br) can then be easily cyclized under basic conditions which include, but are not limited to KOtBu in THF from −78° C. to 25° C., DBU in THF from 0° C. to 75° C. or KN(TMS)₂ in THF from −78° C. to 25° C. In class of the invention, when Y is fluoro, the reaction is performed in the presence of an organic base, wherein the organic base is KN(TMS)₂ in THF; BBr₃ in CH₂Cl₂ followed by KOtBu in THF; or DBU in THF. In the case where Y is a protected hydroxyl group, the protecting group is first removed by conventional means known in the art and then the hydroxyl group is converted to a reactive leaving group such as methanesulfonyloxy (MsCl, Et₃N, CH₂Cl₂), p-toluenesulfonyloxy (TsCl, pyridine, DMAP, CH₂Cl₂) or iodo (i. MsCl, Et₃N, CH₂Cl₂; ii. NaI, acetone). Cyclization is then accomplished as described above for Y═Br.

The bridged tetrahydrofluorenone of formula V is then halogenated to yield a compound of formula I or a compound of formula I with protecting groups attached. In an embodiment of the invention, the enone bond of the bridged tetrahydrofluorenone of formula V is halogenated with a halogenating agent which is NCS in DMF; NBS in DMF; bromine and NaHCO₃ in CH₂Cl₂; or 12 and pyridine in CH₂Cl₂. In a class of the embodiment, the halogenation is performed with NCS in DMF from 0° C. to 60° C., NBS in DMF from 0° C. to 60° C., bromine and NaHCO₃ in CH₂Cl₂, or 12 and pyridine in CH₂Cl₂. The present invention also embodies the introduction of additional R¹ groups via a palladium catalyzed cross-coupling reaction such as a Stille reaction or a Suzuki reaction on a compound V where R¹=Br or I. For introduction of R¹=aryl or heteroaryl suitable conditions are R¹B(OH)₂, CsCO₃, PdCl₂(PPh₃)₂, DMF, 20° C. to 100° C. For introduction of R¹=alkyl, alkenyl or alkynyl, suitable conditions are Bu₃SnR¹, PdCl₂(PPh₃)₂, PhMe, 20° C. to 100° C.

After introduction of the R¹ substituent, a final deprotection step may be required to yield the final product, a compound of formula I. Suitable reagents for deprotection are known to those skilled in the art.

Also provided in this invention are processes for preparing a compound of formula II: 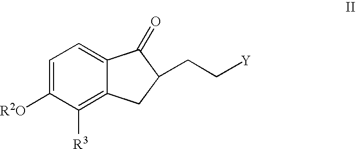
comprising the steps of:
a) Reacting a 5-alkoxy-1-indanone of formula VI with a carboxylating to form a beta-ketoester of formula VII; 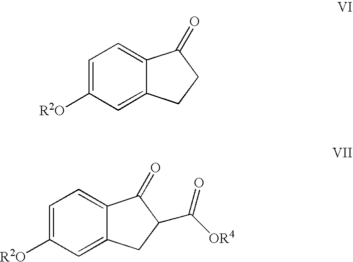
b) Alkylating the beta-ketoester of formula VII to form an alkylated beta-ketoester of formula VIII; 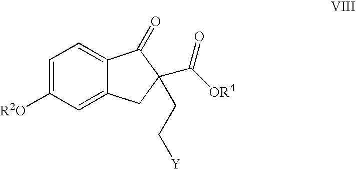
c) Reacting the alkylated ester of formula VIII with an electrophilic reagent to form an intermediate of formula IX; 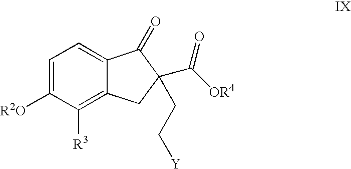
d) Hydrolyzing and decarboxylating the intermediate of formula IX to yield the compound of formula II;
   wherein R² is hydrogen, R^{a}, (C═O)R^{a}, (C═O)OR^{a} or a protecting group for a phenolic hydroxyl;
   R³ is hydrogen, fluoro, chloro, bromo, iodo, C₁₋₂ alkyl, cyano or OR^{a};
   R⁴ is methyl, ethyl, allyl or benzyl;
   Y is fluoro, chloro, bromo, iodo, methanesulfonyloxy, p-toluenesulfonyloxy, trifluoromethanesulfonyloxy, or a precursor thereof;
   R^{a} is hydrogen, C₁₋₄ alkyl or phenyl.

In an embodiment of the invention, R² is hydrogen, R^{a}, (C═O)R^{a}, (C═O)OR^{a} or a protecting group for a phenolic hydroxyl.

A 5-alkoxy-1-indanone of formula VI is reacted with a carboxylating reagent to form a beta-ketoester of formula VII. The 5-alkoxy-1-indanone starting materials are either known compounds or can be prepared by conventional methods known to those skilled in the art. In an embodiment of the invention, suitable carboxylating agents include, but are not limited to, ethyl cyanoformate, ethyl chloroformate, dimethyl carbonate and diethyl carbonate. This reaction can be run in the presence of a base. Suitable bases include, but are not limited to, LDA, LiN(TMS)₂, and sodium hydride.

The beta-ketoester of formula VII is then alkylated in the presence of a base to form an alkylated ester of formula VIII. Suitable alkylating agents include, but are not limited to, BrCH₂CH₂F, ICH₂CH₂F, TfOCH₂CH₂F, ICH₂CH₂Cl and ICH₂CH₂OBn. Suitable bases include, but are not limited to, potassium carbonate, KOt-Bu, sodium hydride and potassium hydride.

To form the intermediate of formula IX, the alkylated beta-ketoester of formula VIII is reacted with a suitable electrophilic reagent which includes, but is not limited to, NCS in DMF from 0° C. to 60° C., NBS in DMF from 0° C. to 60° C., Accufluor™ NFTh, MeCN, 50° C. to 80° C. This electrophilic aromatic substitution may be followed by a transition metal catalyzed cross-coupling reaction such as a Stille reaction to facilitate the introduction of certain groups. For introduction of R³=Me suitable conditions are SnMe₄, PdCl₂(PPh₃)₂, DMF, 20° C. to 120° C.

The intermediate of formula IX is hydrolyzed and decarboxylated to yield a compound of formula II. Suitable reagents for the hydrolysis and decarboxylation include, but are not limited to, NaOH, H₂O, MeOH, 0° C. to 50° C.; 6N HCl, HOAc, 60° C. to 100° C.; LiCl, DMF, 100° C. to 150° C.; BBr₃, CH₂Cl₂, −78° C. to 0° C.

The term “alkyl” shall mean a substituting univalent group derived by conceptual removal of one hydrogen atom from a straight or branched-chain acyclic saturated hydrocarbon (i.e., —CH₃, —CH₂CH₃, —CH₂CH₂CH₃, —CH(CH₃)₂, —CH₂CH₂CH₂CH₃, —CH₂CH(CH₃)₂, —C(CH₃)₃, etc.).

The term “alkenyl” shall mean a substituting univalent group derived by conceptual removal of one hydrogen atom from a straight or branched-chain acyclic unsaturated hydrocarbon (i.e., —CH═CH₂, —CH═CHCH₃, —C═C(CH₃)₂, —CH₂CH═CH₂, etc.).

The term “alkynyl” shall mean a substituting univalent group derived by conceptual removal of one hydrogen atom from a straight or branched-chain acyclic unsaturated hydrocarbon containing a carbon-carbon triple bond (i.e., —C≡CH, —C—CCH₃, —C≡CCH(CH₃)₂, —CH₂C≡CH, etc.).

The term “alkylidene” shall mean a substituting bivalent group derived from a straight or branched-chain acyclic saturated hydrocarbon by conceptual removal of two hydrogen atoms from the same carbon atom (i.e., ═CH₂, ═CHCH₃, ═C(CH₃)₂, etc.).

The term “cycloalkyl” shall mean a substituting univalent group derived by conceptual removal of one hydrogen atom from a saturated monocyclic hydrocarbon (i.e., cyclopropyl, cyclobutyl, cyclopentyl, cyclohexyl, or cycloheptyl).

The term “aryl” as used herein refers to a substituting univalent group derived by conceptual removal of one hydrogen atom from a monocyclic or bicyclic aromatic hydrocarbon. Examples of aryl groups are phenyl, indenyl, and naphthyl.

The term “heteroaryl” as used herein refers to a substituting univalent group derived by the conceptual removal of one hydrogen atom from a monocyclic or bicyclic aromatic ring system containing 1, 2, 3, or 4 heteroatoms selected from N, O, or S. Examples of heteroaryl groups include, but are not limited to, pyrrolyl, furyl, thienyl, imidazolyl, pyrazolyl, oxazolyl, isoxazolyl, thiazolyl, pyridyl, pyrimidinyl, pyrazinyl, benzimidazolyl, indolyl, and purinyl. Heteraryl substituents can be attached at a carbon atom or through the heteroatom.

The term “halo” shall include iodo, bromo, chloro and fluoro.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

In the schemes and examples below, various reagent symbols and abbreviations have the following meanings:

AlCl₃: Aluminum chloride

BBr₃: Boron Tribromide

BrCH₂CH₂F: 1-bromo-2-fluoroethane

BrCH₂CH₂OBn: 1-bromo-2-benzyloxyethane

CH₂Cl₂: Dichloromethane

DBN: 1,5-diazabicyclo[4.3.0]non-5-ene

DBU: 1,8-diazabicyclo[5.4.0]undec-7-ene

DMAC: N,N-Dimethylacetamide

DMF: Dimethylformamide

EtOH: Ethanol

Et₃N: Triethylamine

EtSH: ethanethiol

EVK: Ethyl vinyl ketone

HCl: Hydrochloric acid

HOAc: Acetic Acid

K₂CO₃: Potassium carbonate

KI: Potassium iodide

KN(TMS)₂: Potassium bis(trimethylsilyl)amide

LiCl: Lithium chloride

LDA: Lithium Dimethylamide

LiN(TMS)₂: Lithium bis(trimethylsilyl)amide

Me₂CO₃: Methyl carbonate

MeCN: Acetonitrile

MeOH: Methanol

MsCl: Mesyl chloride

MVK: Methyl vinyl ketone

NaH: Sodium hydride

NaI: Sodium iodide

NaOH: Sodium hydroxide

NaOMe: Sodium methylate

NCCO₂Et: Ethyl cyanoformate

NBS: N-Bromo Succinimide

NCS: N-Chloro Succinimide

PdCl₂(PPh₃)₂: Bis(triphenylphosphine)palladium(II) chloride

Pd(PPh₃)₄: Tetrakis(triphenylphosphine)palladium(0)

PhB(OH)₂: Phenyl borohydride

PhCH₃: Toluene

PhH: Benzene

PhMe: Toluene

SnMe₄: tetramethyltin

THF: Tetrahydrofuran

The compounds of the present invention can be prepared according to the following general scheme, using appropriate materials, and are further exemplified by the subsequent specific examples. The compounds illustrated in the examples are not, however, to be construed as forming the only genus that is considered as the invention. Those skilled in the art will readily understand that known variations of the conditions and processes of the following preparative procedures can be used to prepare these compounds. All temperatures are degrees Celsius unless otherwise noted.

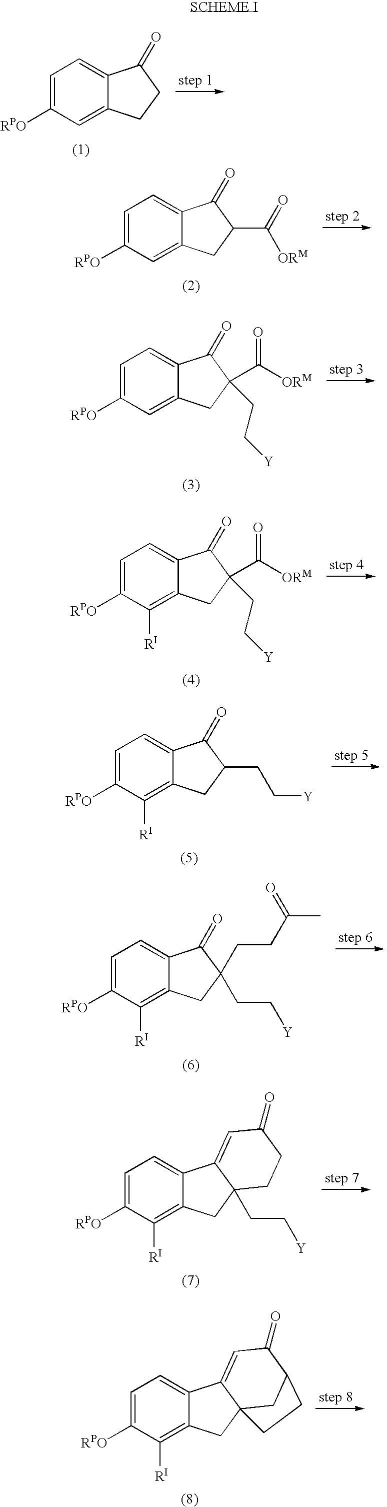

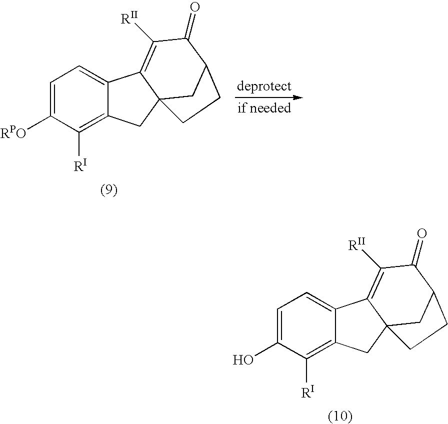

Representative reagents and reaction conditions indicated in Scheme I as steps 1-8 are as follows:

| Step 1 | i) | LiN(TMS)₂, THF, −78 to 40° C. | |
|---|---|---|---|
| | ii) | NCCO₂Et, −78° C. to rt | R^{M} = Et |
| | Me₂CO₃, NaH, PhH, 60° C. | | R^{M} = Me |
| Step 2 | BrCH₂CH₂F, K₂CO₃, KI, DMAC, 65° C. | | Y = F |
| | BrCH₂CH₂OBn, K₂CO₃, KI, DMAC, 60-100° C. | | Y = OBn |
| Step 3 | NCS, DMF, 50° C. | | R^{I} = Cl |
| | NBS, DMF, rt to 50° C. | | R^{I} = Br |
| | Accufluor ™ NFTh, MeCN, 50 to 80° C. | | R^{I} = F |
| | i) | NBS, DMF, rt to 50° C. | R^{I} = Me |
| | ii) | SnMe₄, PdCl₂(PPh₃)₂, DMF, rt to 100° C. | |
| Step 4 | NaOH, H₂O, MeOH, THF 0 to 40° C. or | | |
| | 6N HCl, HOAc, 90-100° C., | | |
| Step 5 | MVK, NaOMe, MeOH, rt to 60° C. or | | |
| | MVK, DBN, THF, rt to 60° C. | | |
| Step 6 | pyrrolidine, HOAc, THF or PhMe, 60-85° C. or | | |
| | NaOH, H₂O, MeOH or EtOH, rt to 85° C. or | | |
| | 6N HCl, HOAc, 90-100° C. | | |
| Step 7 | LiCl, DMF, 150° C. | | Y = F |
| | i) | BBr₃, CH₂Cl₂, −78° C. | Y = F |
| | ii) | KN(TMS)₂, THF, −78° C. | |
| | pyridine-HCl, 190° C. | | Y = OBn |
| | i) | NaOMe, MeOH | Y = OAc |
| | ii) | MsCl, Et₃N, CH₂Cl₂ | |
| | iii) | LDA, THF, −78° C. to rt | |
| Step 8 | NCS, DMF, 50° C. | | R^{II} = Cl |
| | NBS, DMF, rt to 50° C. | | R^{II} = Br |
| | i) | NBS, DMF, rt to 50° C. | R^{II} = Ph |
| | ii) | PhB(OH)₂, Pd(PPh₃)₄, PhCH₃, rt to 100° C. | |

Scheme II illustrates a variation of the synthesis shown in Scheme I. In this variation, the starting indanone (1a) is already substituted with the R^{I} substitutent at position 4. Indanones (1a) are either known compounds or can be prepared by conventional methods known in the art. In step 1 of Scheme II, the indanone (1a) is substituted at the 2-position with the moiety —CH₂CH₂—Y. This can be accomplished by a reductive alkylation reaction wherein (1a) is reacted with a substituted aldehyde Y—CH₂CHO under basic conditions followed by hydrogenation of the resulting alkylidene intermediate. In this instance Y is most appropriately a precursor group which can be converted to a displaceable leaving group. Alternatively, introduction of the moiety —CH₂CH₂—Y can be accomplished by reacting indanone (1a) with an alkylating agent Z-CH₂CH₂—Y, where Z represents a displaceable leaving group, in the presence of a base to give intermediate (2). In the case where Y also represents a displaceable leaving group, the relative reactivities of the two groups are appropriately chosen so that Z is the more easily displaced group. Step 2 in Scheme II is analogous to step 5 of Scheme I, but employs the substituted vinyl ketone CH₂CH₂COCH₂R^{II} in place of methyl vinyl ketone. Diketone (11) is then converted to (10a) by the procedures previously described in Scheme I except that a separate step to introduce the R^{II} substituent is not required since it is incorporated in step 2 of Scheme II.

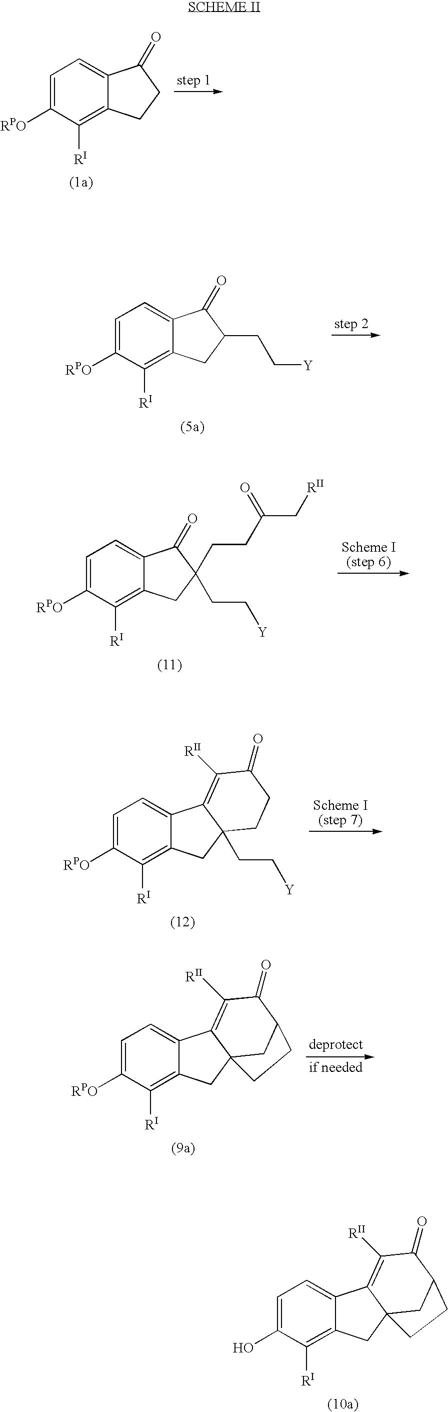

Representative reagents and reaction conditions indicated in Scheme II as steps 1-2 are as follows:

| | | |
|---|---|---|
| Step 1 | BnOCH₂CHO, NaOMe, MeOH, H₂, Pd/C | Y = OBn |
| | (HOCH₂CHO)₂, NaOMe, MeOH, H₂, Pd/C | Y = OH |
| Step 2 | CH₂═CHC(O)CH₂R^{II}, NaOMe, MeOH, rt | |
| | to 60° C. or | |
| | CH₂═CHC(O)CH₂R^{II}, DBN, THF, rt to 60° C. | |

Scheme III illustrates a variation of the synthesis shown in Scheme II which allows for introduction of the R^{III} substituent. Step 1 of Scheme III is similar to step 1 of Scheme II except that the reduction step is omitted and the alkylidene intermediate (13) is obtained. Introduction of the R^{III} substituent is accomplished in step 2 by reaction of (13) with an appropriate organometallic species to give (14) via a 1,4-conjugate addition reaction. Indanone (14) is then converted to (10b) by the procedures previously described in Scheme I.

### EXAMPLE 1

### SYNTHESIS of (7-BETA,9a-BETA)-1,5-DICHLORO-2-HYDROXYGIBBA-1,3,4a(10a),4b-TETRAEN-6-ONE

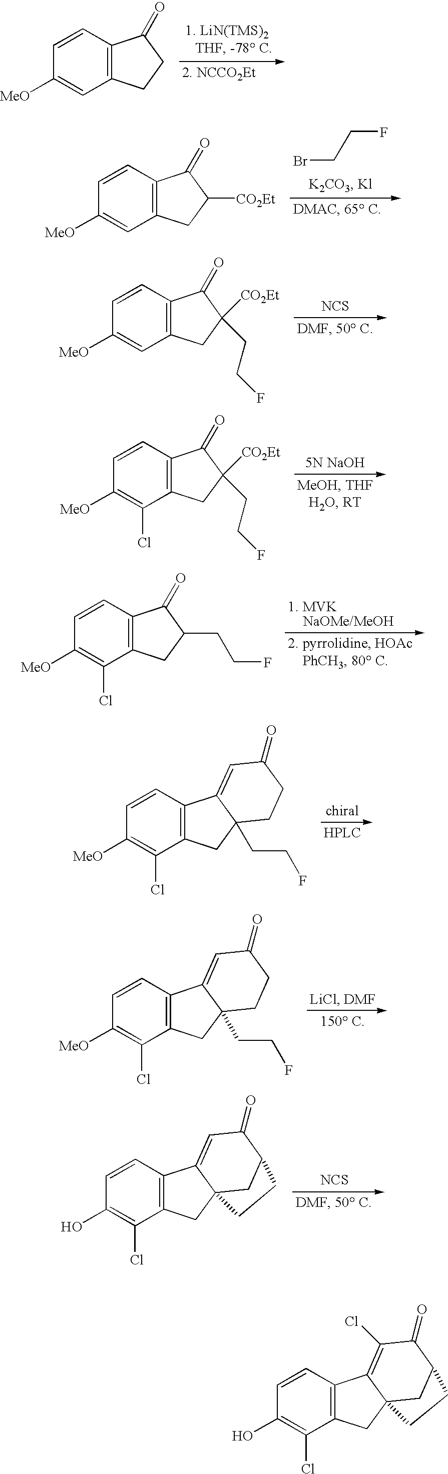

### Step 1: ethyl 5-methoxy-1-oxoindane-2-carboxylate

To a solution of 5-methoxyindan-1-one (15.0 g, 92.5 mmol) in THF (370 mL) at −78° C. was added a 1.0 M solution of lithium bis(trimethylsilyl)amide in TB (200 mL, 200 mmol) via an addition funnel during 15 minutes. After 40 minutes, ethyl cyanoformate (14.0 mL, 142 mmol) was added during several minutes and the reaction mixture was allowed to warm gradually. After 30 minutes, the reaction mixture was partitioned between EtOAc and dilute aqueous HCl and the organic phase was washed with water and brine and dried over Na₂SO₄. Filtration and removal of the solvent under reduced pressure gave ethyl 5-methoxy-1-oxoindane-2-carboxylate as a brown solid which was used in the next step without purification.

The reaction was repeated starting with 15.82 g (97.5 mmol) of 5-methoxyindan-1-one to give additional crude ethyl 5-methoxy-1-oxoindane-2-carboxylate.

### Step 2: ethyl 2-(2-fluoroethyl)-5-methoxy-1-oxoindane-2-carboxylate

To a mixture of ethyl 5-methoxy-1-oxoindane-2-carboxylate (crude product from the preceding two reactions, ˜190 mmol), K₂CO₃ (53.8 g, 389 mmol) and KI (64.7 g, 390 mmol) in anhydrous dimethylacetamide (792 mL) was added 1-bromo-2-fluoroethane (18.4 mL, 247 mmol) and the mixture was stirred and heated at 65° C. After 20 hours, analysis of an aliquot by NMR showed the reaction to be complete. After cooling to room temperature, most of the dimethylacetamide was removed by evaporation at reduced pressure. The residue was partitioned between EtOAc and water and the organic phase was washed with water (4 times) and brine and dried over Na₂SO₄. Filtration and removal of the solvent under reduced pressure gave crude ethyl 2-(2-fluoroethyl)-5-methoxy-1-oxoindane-2-carboxylate which was used in the next step without purification.

### Step 3: ethyl 4-chloro-2-(2-fluoroethyl)-5-methoxy-1-oxoindane-2-carboxylate

To a solution of ethyl 2-(2-fluoroethyl)-5-methoxy-1-oxoindane-2-carboxylate (44.6 g, 159 mmol) in DMF (159 mL) was added N-chlorosuccinimide (23.4 g, 175 mmol) in portions. The solution was heated at 50° C. and the reaction was monitored periodically by NMR analysis of aliquots. After 6 hours, the reaction was approximately 80% complete by NMR analysis. The reaction mixture was allowed to cool to room temperature and stand overnight. After reheating to 50° C., additional N-chlorosuccinimide (2.12 g, 15.9 mmol) was added. Monitoring by NMR was continued, and after 4.5 hours another portion of N-chlorosuccinimide (2.12 g, 15.9 mmol) was added. After another 3 hours, the reaction was allowed to cool to room temperature and stand overnight. Most of the DMF was removed by evaporation at reduced pressure and the residue was partitioned between EtOAc and water. The organic phase was washed with water (4 times) and brine and dried over Na₂SO₄. Filtration and removal of the solvent under reduced pressure gave crude ethyl 4-chloro-2-(2-fluoroethyl)-5-methoxy-1-oxoindane-2-carboxylate. This material was used in the next step without purification.

### Step 4: 4-chloro-2-(2-fluoroethyl)-5-methoxyindan-1-one

To a solution of ethyl 4-chloro-2-(2-fluoroethyl)-5-methoxy-1-oxoindane-2-carboxylate (56.4 g of crude product from the previous reaction) in THF (330 mL) was added methanol (50 mL) followed by a solution of methanol (116 mL)/water (166 mL). To the resulting clear red-orange solution was added 5N aqueous NaOH (55.7 mL, 279 mmol) gradually during 9 minutes giving a black solution. After 3.5 hours, the reaction was quenched by addition of 12N aqueous HCl (30 mL, 360 mmol) and most of the THF and methanol were removed by rotary evaporation at reduced pressure. The residue was partitioned between EtOAc and water and the organic phase was washed with saturated aqueous NaHCO₃ and brine and dried over MgSO₄. Filtration and removal of the solvent under reduced pressure gave crude product. Purification by flash chromatography on silica gel (elution with CH₂Cl₂) gave the product. Re-purification of mixed fractions gave additional product. The combined yield was 4-chloro-2-(2-fluoroethyl)-5-methoxyindan-1-one which by NMR contained approximately 4% of the undesired 6-chloro-2-(2-fluoroethyl)-5-methoxyindan-1-one regioisomer.

### Step 5: 8-chloro-9a-(2-fluoroethyl)-7-methoxy-1,2,9,9a-tetrahydro-3H-fluoren-3-one

To a suspension of 4-chloro-2-(2-fluoroethyl)-5-methoxyindan-1-one (18.0 g, 74.2 mmol) in methanol (250 mL) was added methyl vinyl ketone (7.7 mL, 92 mmol) during 2 minutes followed by addition of a 0.5 M solution of sodium methoxide in methanol (74.2 mL, 37.1 mmol). After 3 hours at room temperature, analysis of an aliquot by NMR and LC/MS showed the reaction to be complete. The dark reaction mixture was concentrated by rotary evaporation under reduced pressure. The residual oil was dissolved in toluene (980 mL) and acetic acid (6.4 mL, 112 mmol) was added followed by pyrrolidine (6.2 mL, 74.2 mmol). The resulting solution was heated at 80° C. for 3.25 hours and was then allowed to cool to room temperature and stand overnight. The reaction mixture was partitioned between EtOAc and water and the organic phase was washed successively with dilute aqueous HCl, dilute aqueous NaHCO₃ and brine. After drying over MgSO₄, filtration and evaporation gave crude product. Purification by flash chromatography on a column of 400 g of silica gel (elution with 5% EtOAc/CH₂Cl₂) gave the product. Re-purification of some impure fractions gave additional product. The combined yield was 8-chloro-9a-(2-fluoroethyl)-7-methoxy-1,2,9,9a-tetrahydro-3H-fluoren-3-one which by NMR contained approximately 4% of the undesired 6-chloro-9a-(2-fluoroethyl)-7-methoxy-1,2,9,9a-tetrahydro-3H-fluoren-3-one regioisomer.

### Step 6: Resolution of racemic 8-chloro-9a-(2-fluoroethyl)-7-methoxy-1,2,99a-tetrahydro-3H-fluoren-3-one by chiral HPLC

Racemic 8-chloro-9a-(2-fluoroethyl)-7-methoxy-1,2,9,9a-tetrahydro-3H-fluoren-3-one (17 g) was resolved by chiral HPLC on a Daicel Chiralcel OD column (elution with 15% EtOH:Heptane, fractions monitored at 220 nm). The pure fractions containing the first enantiomer to elute were combined and concentrated to give (9aR)-8-chloro-9a-(2-fluoroethyl)-7-methoxy-1,2,9,9a-tetrahydro-3H-fluoren-3-one as an oil which had a positive rotation. The fractions containing the second enantiomer to elute were combined and concentrated to give of (9aS)-8-chloro-9a-(2-fluoroethyl)-7-methoxy-1,2,9,9a-tetrahydro-3H-fluoren-3-one as an oil which had a negative rotation.

### Step 7: (7beta,9abeta)-1-chloro-2-hydroxygibba-1,3,4a(10a),4b-tetraen-6-one

To a mixture of (9aS)-8-chloro-9a-(2-fluoroethyl)-7-methoxy-1,2,9,9a-tetrahydro-3H-fluoren-3-one (5.34 g, 18.1 mmol) and lithium chloride (7.68 g, 181 mmol) was added DMF (102 mL) and the stirred suspension was heated to 150° C. giving a yellow solution. After 21 hours, the solution was cooled to room temperature and partitioned between EtOAc and 0.2N aqueous HCl. The organic phase was washed with water (4 times) and brine and dried over MgSO₄. Filtration and evaporation gave crude product. Purification by flash chromatography on silica gel (elution with 20% EtOAc/CH₂Cl₂) gave (7beta,9abeta)-1-chloro-2-hydroxygibba-1,3,4a(10a),4b-tetraen-6-one.

### Step 8: (7beta,9abeta)-1,5-dichloro-2-hydroxygibba-1,3,4a(10a),4b-tetraen-6-one

To a solution of (7beta,9abeta)-1-chloro-2-hydroxygibba-1,3,4a(10a),4b-tetraen-6-one (3.51 g, 13.5 mmol) in DMF (54 mL) was added N-chlorosuccinimide (1.8 g, 13.5 mmol) and the reaction mixture was heated to 50° C. After 3 hours, NMR analysis of an aliquot showed the reaction to be complete. The reaction mixture was cooled to room temperature and partitioned between EtOAc and dilute aqueous HCl. The organic phase was washed with water (4 times) and brine and dried over MgSO₄. Filtration and evaporation gave crude product. Purification by flash chromatography was accomplished by pre-adsorbing a solution of the crude product in MeOH(CH₂Cl₂ onto silica gel. Elution of the column with 20% to 35% EtOAc/CH₂Cl₂ gave the product as a solid which was dissolved in ethanol and precipitated with water. Filtration and evaporation under vacuum gave (7beta,9abeta)-1,5-dichloro-2-hydroxygibba-1,3,4a(10a),4b-tetraen-6-one as a pale yellow powder.

¹H NMR (CDCl₃, 500 MHz); δ 1.74-1.80 (m, 1H), 1.96-1.99 (m, 2H), 2.03 (dd, 1H), 2.13 (d, 1H), 2.33-2.40 (m, 1H), 3.17 (d, 1H), 3.25-3.30 (m, 1H), 3.32 (d, 1H), 6.01 (s, 1H), 7.09 (d, 1H), 8.25 (d, 1H).

Mass spectrum: (ESI) m/z=295 (M+H).

### EXAMPLE 2

### SYNTHESIS OF 2-HYDROXY-5-METHYLGIBBA-1,3,4a(10a),4b-TETRAEN-6-ONE

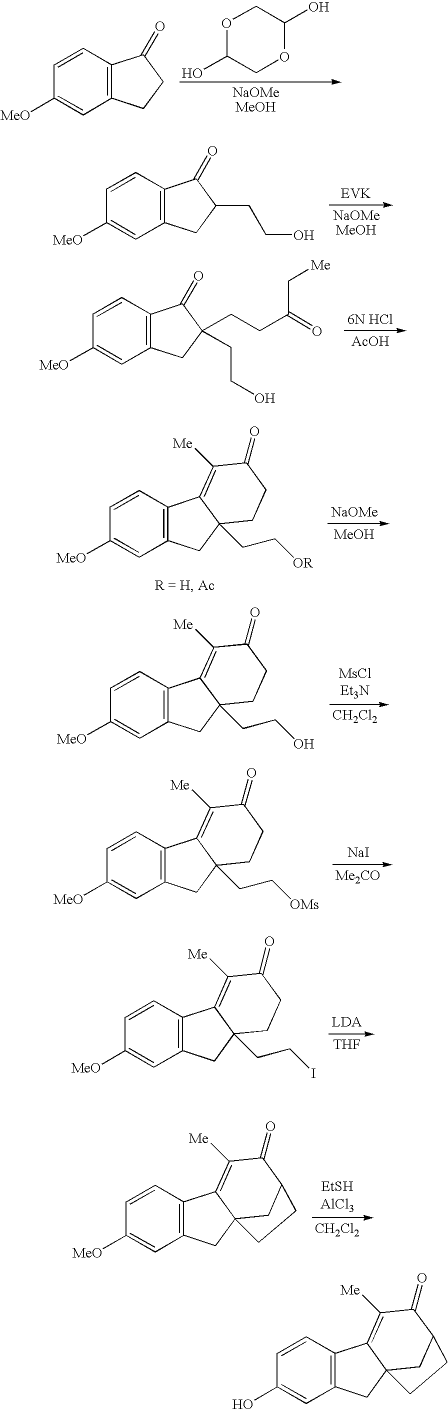

### Step 1: 2-(2-hydroxyethyl)-5-methoxy-1-indanone

A solution of 5-methoxy-1-indanone (500 mg, 3.08 mmol) in methanol (10 mL) was treated with 10% palladium on carbon (53 mg) followed by glycoaldehyde dimer (370 mg, 3.08 mmol) and 0.5M sodium methoxide in methanol (1.3 mL, 0.65 mmol). The mixture was placed under a hydrogen atmosphere (balloon) and stirred vigorously at room temperature for 65 hours. After purging with nitrogen, the mixture was filtered through a 0.45 μm Acrodisc and the disk was rinsed with methanol (2 mL). The filtrate was diluted with EtOAc (25 mL), washed with 0.1N HCl (15 mL) and brine (15 mL), dried over MgSO₄, filtered, and evaporated under vacuum to a solid. LC-MS of this material showed a mixture of starting material (major) and product.

The mixture was purified by chromatography on a Biotage Flash 12M KP-Sil column (12 mm×15 cm). The column was eluted with 3:2 EtOAc-hexanes, collecting 6 mL fractions every 30 sec. Fractions 20-36 were concentrated under vacuum and flashed with benzene to afford 2-(2-hydroxyethyl)-5-methoxy-1-indanone as an oil.

¹H NMR (CDCl₃, 500 MHz) δ 1.80 and 2.05 (two m, CH₂CH₂OH), 2.79 and 3.35 (two dd, 3-CH₂), 2.83 (m, H-2), 3.77-3.90 (m, CH₂CH₂OH), 3.87 (s, OCH₃), 6.86 (d, H-4), 6.89 (dd, H-6), and 7.67 (d, H-7).

### Step 2: 2-(2-hydroxyethyl)-5-methoxy-2-(3-oxopentyl)-1-indanone

A solution of 2-(2-hydroxyethyl)-5-methoxy-1-indanone (105 mg, 0.51 mmol) in methanol (2.0 mL) at room temperature was treated with ethyl vinyl ketone (EVK, 0.102 mL) and 0.5M sodium methoxide in methanol (0.204 mL, 0.1 mmol). The mixture was stirred in a capped flask and heated in an oil bath at 60° C. for 8 hours. After cooling, the reaction mixture was diluted with EtOAc (25 mL), washed with 0.2N HCl (15 mL), water (15 mL), and brine (15 mL), dried over MgSO₄, filtered, and evaporated under vacuum to afford 2-(2-hydroxyethyl)-5-methoxy-2-(3-oxopentyl)-1-indanone as an oil.

¹H NMR (CDCl₃, 500 MHz) δ 0.99 (t, COCH₂CH₃), 1.84-2.00 (m, CH₂CH₂OH and CH₂CH₂CO), 2.28 (m, CH₂CH₂CO), 2.33 (m, COCH₂CH₃), 2.92 and 3.11 (two d, 3-CH₂), 3.63 and 3.72 (two m, CH₂CH₂OH), 3.87 (s, OCH₃), 6.86 (d, H-4), 6.91 (dd, H-6), and 7.67 (d, H-7).

### Step 3: 9a-(2-hydroxyethyl)-7-methoxy-4-methyl-1,2,9,9a-tetrahydro-3H-fluoren-3-one and 9a-(2-acetoxyethyl)-7-methoxy-4-methyl-1,2,9,9a-tetrahydro-3H-fluoren-3-one

A solution of 2-(2-hydroxyethyl)-5-methoxy-2-(3-oxopentyl)-1-indanone (138 mg, 0.475 mmol) in acetic acid (3.0 mL) was diluted with aqueous 6N HCl (3.0 mL) and the resulting mixture was stirred and heated in an oil bath at 80° C. for 90 minutes. After cooling to room temperature, the reaction mixture was diluted with EtOAc (20 mL), washed with water (10 mL), 1M pH 7 phosphate buffer (15 ml), water (15 mL), and brine (15 mL), dried over MgSO₄, filtered, and evaporated under vacuum to an oil. LC-MS showed a mixture of 9a-(2-hydroxyethyl)-7-methoxy-4-methyl-1,2,9,9a-tetrahydro-3H-fluoren-3-one and its O-acetyl derivative 9a-(2-acetoxyethyl)-7-methoxy-4-methyl-1,2,9,9a-tetrahydro-3H-fluoren-3-one.

### Step 4: 9a-(2-hydroxyethyl)-7-methoxy-4-methyl-1,2,9,9a-tetrahydro-3H-fluoren-3-one

The mixture of products from step 3 was dissolved in methanol (5 mL) and the solution treated with 0.5M sodium methoxide in methanol (4.5 mL). The mixture was stirred at room temperature for 15 minutes then acidified with aqueous 2N HCl and concentrated under vacuum. The residue in EtOAc (25 mL) was washed with brine (20 mL), dried over MgSO₄, filtered, and evaporated under vacuum. The crude product was purified by chromatography on a Biotage Flash-12 M KP-Sil column (12 mm×15 cm). The column was eluted with 3:2 EtOAc-hexanes (145 mL) followed by 100% EtOAc, collecting 4 mL fractions every 30 seconds. Fractions 30-50 were combined and evaporated under vacuum to give the product as an oil. Treatment of this material with Et₂O gave the product 9a-(2-hydroxyethyl)-7-methoxy-4-methyl-1,2,9,9a-tetrahydro-3H-fluoren-3-one as a solid.

¹H NMR (CDCl₃, 500 MHz) δ 1.72-1.86 (m, CH₂CH₂OH), 1.99 and 2.21 (two ddd, 1-CH₂), 2.04 (s, 4-CH₃), 2.45 and 2.63 (two ddd, 2-CH₂), 2.76 and 3.05 (two d, 9-CH₂), 3.47-3.62 (m, CH₂CH₂OH), 3.82 (s, OCH₃), 6.81-8.85 (m, H-6 and H-8), and 7.61 (d, H-5).

### Step 5: 9a-[2-(methanesulfonyoxy)ethyl]-7-methoxy-4-methyl-1,2,9,9a-tetrahydro-3H-fluoren-3-one

An ice-cold solution of 9a-(2-hydroxyethyl)-7-methoxy-4-methyl-1,2,9,9a-tetrahydro-3H-fluoren-3-one (39 mg, 0.14 mmol) and triethylamine (0.030 mL, 0.21 mmol) in anhydrous dichloromethane (1.5 ml) was treated with methanesulfonyl chloride (0.014 mL, 0.18 mmol) and the resulting solution was stirred at 0° C. for 30 minutes. The mixture was diluted with EtOAc (10 mL), washed with water (5 mL), 0.2N HCl (5 mL), and brine (5 mL), dried over MgSO₄, filtered, and evaporated under vacuum to provide 9a-[2-(methanesulfonyoxy)ethyl]-7-methoxy-4-methyl-1,2,9,9a-tetrahydro-3H-fluoren-3-one as an oil.

¹H NMR (CDCl₃, 500 MHz) δ 2.03 (m, CH₂CH₂O), 2.08 (s, 4-CH₃), 2.09 and 2.22 (two ddd, 1-CH₂), 2.53 and 2.61 (two ddd, 2-CH₂), 2.85 and 3.03 (two d, 9-CH₂), 2.89 (s, SO₂CH₃), 3.85 (s, OCH₃), 4.03-4.17 (m, CH₂CH₂O), 6.86 (s, H-8), 6.87 (dd, H-6), and 7.64 (d, H-5).

### Step 6: 9a-(2-iodoethyl)-7-methoxy-4-methyl-1,2,9,9a-tetrahydro-3H-fluoren-3-one

A solution of 2-(2-methoxy-5-methyl-6-oxo-6,7,8,9-tetrahydro-8a-H-fluoren-8a-yl)ethyl methanesulfonate (49.7 mg, 0.142 mmol) in acetone (2.0 mL) was treated with sodium iodide (85 mg, 0.57 mmol) and the resulting mixture was stirred and heated in an oil bath at 60° C. for 16 hours. After cooling, the mixture was diluted with acetone (2 mL) and filtered through a 0.45 μm Acrodisc filter. The filtrate was evaporated under vacuum and the residue in CH₂Cl₂ (3 mL) was re-filtered. The filtrate was purified by chromatography on a Biotage Flash 12M KP-Sil column (12 mm×15 cm) which was eluted with 4:1 hexanes-EtOAc, collecting 6 mL fractions every 30 seconds. Fractions 9-11 gave 9a-(2-iodoethyl)-7-methoxy-4-methyl-1,2,9,9a-tetrahydro-3H-fluoren-3-one as an oil.

¹H NMR (CDCl₃, 500 MHz) δ 2.03 and 2.20 (two ddd, 1-CH₂), 2.08 (s, 4-CH₃), 2.24 (m, CH₂CH₂I), 2.51 and 2.61 (two ddd, 2-CH₂), 2.80 and 2.97 (two d, 9-CH₂), 2.85 and 2.95 (two m, CH₂CH₂I), 3.86 (s, OCH₃), 6.86 (br s, H-8), 6.87 (dd, H-6), and 7.64 (d, H-5).

### Step 7: 2-methoxy-5-methylgibba-1,3,4a(10a),4b-tetraen-6-one

A solution of N,N-diisopropylamine (0.015 mL, 0.107 mmol) in anhydrous tetrahydrofuran (THF, 1.0 mL) was placed under a nitrogen atmosphere, cooled in an ice bath, and treated with 1.6 M n-butyllithium in hexanes (0.061 mL, 0.098 mmol). The solution was stirred at 0° C. for 35 minutes, then cooled in a dry ice-acetone bath and, after aging for 5 minutes, treated with a solution of 9a-(2-iodoethyl)-7-methoxy-4-methyl-1,2,9,9a-tetrahydro-3H-fluoren-3-one (34 mg, 0.089 mmol) in THF (1.0 mL). The reaction mixture was warmed from −78° C. to room temperature over 4 hours, stirred at room temperature for 21 hours, and then quenched with aqueous 2N HCl (0.5 mL) and diluted with EtOAc (10 mL). The organic phase was washed with water (5 mL) and brine (5 mL), dried over MgSO₄, filtered, and evaporated under vacuum to a an oil. This material was purified by chromatography on a Biotage Flash 12M KP-Sil column (12 mm×15 cm), eluting with 6:1 hexanes-EtOAc and collecting 7 mL fractions every 30 seconds. Fractions 16-20 were combined and evaporated under vacuum to give a mixture (21.7 mg) of 2-methoxy-5-methylgibba-1(10a),2,4,4b-tetraen-6-one and the starting material 9a-(2-iodoethyl)-7-methoxy-4-methyl-1,2,9,9a-tetrahydro-3H-fluoren-3-one as an oil.

### Step 8: 2-hydroxy-5-methylgibba-1,3,4a(10a),4b-tetraen-6-one

A solution of the product mixture from step 7 (21.7 mg, approx. 0.1 mmol) in anhydrous dichloromethane (1.0 mL) was treated at room temperature with aluminum chloride (75 mg, 0.56 mmol) and ethanethiol (0.032 mL, 0.43 mmol). After stirring at room temperature for 58 minutes, the yellow solution was treated with aqueous 2N HCl (1 mL) and EtOAc (9 mil), washed with water (4 mL) and brine (5 mL), dried over MgSO₄, filtered, and evaporated under vacuum to a solid film. The solid in warm EtOH (1 mL) was applied to two 0.1×20×20 cm silica gel GF plates which were developed with 1:1-hexanes-EtOAc. Two UV visible bands were removed, eluted with EtOAc, concentrated under vacuum, and the residues lyophilized from benzene containing some acetone. The band at R_{f} 0.47-0.57 gave mainly 2-hydroxy-5-methylgibba-1,3,4a(10a),4b-tetraen-6-one as an amorphous solid (contains approx. 16% of the minor 9a-iodoethyl product).

¹H NMR (CDCl₃, 500 MHz): δ 1.63-1.71 (m, 1H), 1.78-1.89 (m, 2H), 1.91 (dd, 1H), 1.98 (d, 1H), 2.09 (s, 3H), 2.24-2.33 (m, 1H), 3.00 (d, 1H), 3.10 (dd, 1H), 3.25 (d, 1H), 5.90 (bs, 1H), 6.86 (dd, 1H), 6.89 (bs, 1H), 7.67 (d, 1H).

What is claimed is:

1. A process for preparing a compound of formula I: 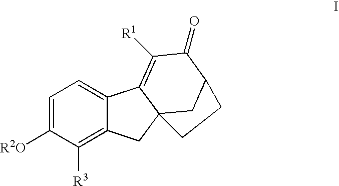
comprising the steps of:
a) Reacting a 2-substituted indanone of formula II with methyl vinyl ketone in the presence of a base to form a diketone of formula III; 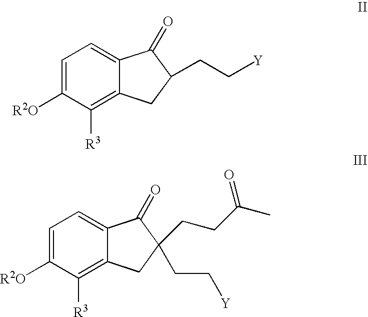
b) Cyclizing the diketone of formula III to form a tetrahydrofluorenone of formula IV; 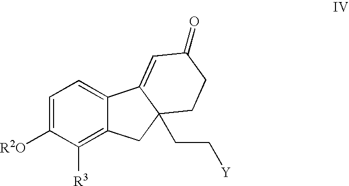
c) Performing an internal alkylation reaction to form a bridged tetrahydrofluorenone of formula V; 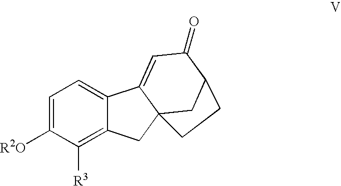
d) Substituting the enone double bond of the bridged tetrahydrofluorenone of formula V to yield the compound of formula I;
wherein R¹ is fluoro, chloro, bromo, iodo, cyano, C₁₋₄ alkyl, C₂₋₄ alkenyl, C₂₋₄ alkynyl, C₃₋₆ cycloalkyl, aryl, or heteroaryl, wherein said alkyl, alkenyl, alkynyl, cycloalkyl, aryl and heteroaryl groups are optionally substituted with one, two or three groups selected from the group consisting of fluoro, chloro, bromo, iodo, cyano and OR^{a};
R² is hydrogen, R^{a}, (C═O)R^{a} or (C═O)OR^{a};
R³ is hydrogen, fluoro, chloro, bromo, iodo, C₁₋₂ alkyl, cyano or OR^{a};
Y is fluoro, chloro, bromo, iodo, methanesulfonyloxy, p-toluenesulfonyloxy, trifluoromethanesulfonyloxy, or a precursor thereof;
R^{a} is hydrogen, C₁₋₄ alkyl or phenyl.

2. The process of claim 1 wherein the base in step a) is sodium methoxide in methanol, potassium hydroxide in ethanol or DBU in THF.

3. The process of claim 1 wherein cyclizing step b) is performed under basic conditions or acidic conditions.

4. The process of claim 3 wherein the basic conditions are sodium hydroxide in ethanol, sodium methoxide in methanol, or pyrrolidine-acetic acid in toluene.

5. The process of claim 3 wherein the acidic conditions are hydrochloric acid in acetic acid, trifluoroacetic acid, or p-toluenesulfonic acid in toluene.

6. The process of claim 1 wherein step c) is performed with heating, performed in the presence of an organic base or performed in the presence of an organic base with heating.

7. The process of claim 6 wherein Y is fluoro; and step c) is performed in the presence of an organic base with heating, wherein the organic base is LiCl in DMF and heated at 150° C.

8. The process of claim 6 wherein Y is fluoro; and step c) is performed in the presence of an organic base wherein the organic base is KN(TMS)₂ in THF; BBr₃ in CH₂Cl₂ followed by KOtBu in THF; or DBU in THF.

9. The process of claim 1 wherein the enone double bond of the bridged tetrahydrofluorenone of formula V is halogenated to yield the compound of formula I.

10. The process of claim 9 wherein the enone double bond of the bridged tetrahydrofluorenone of formula V is halogenated with a halogenating agent which is NCS in DMF; NBS in DMF; bromine and NaHCO₃ in CH₂Cl₂; or 12 and pyridine in CH₂Cl₂.

11. A process for preparing a compound of formula II: 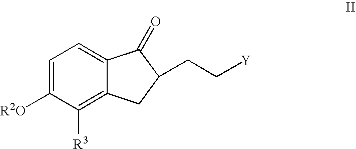
comprising the steps of:
a) Reacting a 5-alkoxy-1-indanone of formula VI with a carboxylating reagent to form a beta-ketoester of formula VII; 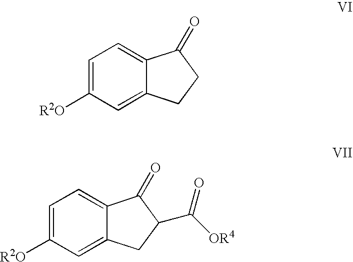
b) Alkylating the beta-ketoester of formula VII to form an alkylated beta-ketoester of formula VIII; 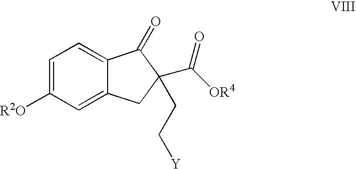
c) Reacting the alkylated beta-ketoester of formula VIII with an electrophilic reagent to form an intermediate of formula IX; 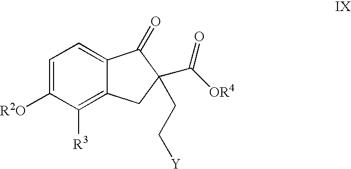
d) Hydrolyzing and decarboxylating the intermediate of formula IX to yield the compound of formula II;
wherein R² is hydrogen, R^{a}, (C═O)R^{a} or (C═O)OR^{a};
R³ is hydrogen, fluoro, chloro, bromo, iodo, C₁₋₂ alkyl, cyano or OR^{a};
R⁴ is methyl, ethyl, allyl or benzyl;
Y is fluoro, chloro, bromo, iodo, methanesulfonyloxy, p-toluenesulfonyloxy, trifluoromethanesulfonyloxy, or a precursor thereof;
R^{a} is hydrogen, C₁₋₄ alkyl or phenyl.
